(19) United States
(12) Patent Application Publication
(10) Pub. No.: US 2016/0256063 A1
(43) Pub. Date: Sep. 8, 2016

(54) ANALYTE ASSESSMENT AND ARRHYTHMIA RISK PREDICTION USING PHYSIOLOGICAL ELECTRICAL DATA

(71) Applicants: Paul A. FRIEDMAN, Rochester, MN (US); Kevin E. BENNET, Rochester, MN (US); Charles J. BRUCE, Rochester, MN (US); Virend K. SOMERS, Rochester, MN (US); Samuel J. ASIRVATHAM, Rochester, MN (US); Michael J. ACKERMAN, Rochester, MN (US); John J. DILLION, Rochester, MN (US); Dan SADOT, Kfar Bilu (IL); Yehu SAPIR, Gedera (IL); Amir GEVA, Tel Aviv (IL); MAYO FOUNDATION FOR MEDICAL EDUCATION AND RESEARCH, Rochester, MN (US)

(72) Inventors: Paul A. Friedman, Rochester, MN (US); Kevin E. Bennet, Rochester, MN (US); Charles J. Bruce, Rochester, MN (US); Virend K. Somers, Rochester, MN (US); Samuel J. Asirvatham, Rochester, MN (US); Michael J. Ackerman, Rochester, MN (US); John J. Dillon, Rochester, MN (US); Dan Sadot, Kfar Bilu (IL); Yehu Sapir, Gedera (IL); Amir Geva, Tel Aviv (IL)

(73) Assignee: Mayo Foundation for Medical Education and Research, Rochester, MN (US)

(21) Appl. No.: 15/025,158

(22) PCT Filed: Sep. 26, 2014
(86) PCT No.: PCT/US14/57811

§ 371 Date: Mar. 25, 2016

Related U.S. Application Data
(60) Provisional application No. 61/883,768, filed on Sep. 27, 2013.
(60) Provisional application No. 61/930,864, filed on Jan. 23, 2014.
(60) Provisional application No. 62/004,737, filed on May 29, 2014.

(51) Int. Cl. A61B 5/0402; A61B 5/04; A61B 5/00; A61B 5/0456; A61B 5/0472; A61B 5/145; A61B 5/0245; A61B 5/0452

(52) U.S. Cl. CPC A61B 5/0402; A61B 5/0245; A61B 5/02455; A61B 5/04011; A61B 5/04012; A61B 5/04525; A61B 5/0456; A61B 5/0472; A61B 5/14546; A61B 5/686; A61B 5/6852; A61B 5/6861

(57) ABSTRACT

This document describes, among other things, a computer-implemented method that includes accessing, by a computer system, electrogram data for a patient, wherein the electrogram data is obtained using one or more leads that sense physiological electrical activity of the patient. The computer system can identify one or more waveform features from the electrogram data, and one or more correlations between values of the one or more waveform features and analyte levels. One or more estimated analyte levels in the patient are determined based on 1) the one or more waveform features identified from the electrogram data and 2) the one or more correlations. The computer system can output information related to the one or more estimated analyte levels.

### CROSS-REFERENCE TO RELATED APPLICATIONS

This application claims the benefit of U.S. Provisional Application Ser. No. 62/004,737, filed May 29, 2014; U.S. Provisional Application Ser. No. 61/930,864, filed Jan. 23, 2014; and U.S. Provisional Application Ser. No. 61/883,768, filed Sep. 27, 2013. The disclosure of the prior applications are considered part of (and are incorporated by reference in their entirety in) the disclosure of this application.

### TECHNICAL FIELD

This document generally describes computer-based technology for analyzing electrocardiogram (ECG) data.

### BACKGROUND

Research has indicated that a potassium change in the blood has an effect on the electrical potential of the heart membrane cells.

### SUMMARY

This document describes computer-based techniques for quantifying the concentration of potassium and other analytes in a patient's blood based on measurements of electrical potentials associated with the patient's body, such as ECG measurements. These techniques can also be used to quantify the concentration of other analytes (such as calcium, magnesium, phosphorous, and others), and to assess drug effects and levels.

In some implementations, a computer-implemented method can include accessing, by a computer system, electrogram data for a patient, wherein the electrogram data are obtained using one or more leads that sense physiological electrical activity of the patient. The computer system can identify one or more waveform features from the electrogram data, and one or more correlations between values of the one or more waveform features and analyte levels. One or more estimated analyte levels in the patient are determined based on 1) the one or more waveform features identified from the electrogram data and 2) the one or more correlations. The computer system can output information related to the one or more estimated analyte levels.

These and other implementations can optionally include one or more of the following features.

The electrogram data may be obtained from one or more physiological electrograms including electrocardiograms (ECG), brain electrograms (EEG), muscular electrograms, myoelectrograms, and neuro-electrograms. The electrogram data may be obtained using surface techniques (e.g., surface ECG), intracardiac techniques, subcutaneous techniques, implanted pacemakers, and defibrillators, for example. In some implementations, electrogram data can include data obtained by measuring electrical activity from the heart by various means.

The method can further include, before identifying the one or more waveform features, filtering the electrogram data to generate filtered electrogram data. The one or more waveform features can be identified from the filtered electrogram data. The filtering can include a first filtering process that includes identifying R peak values in the electrogram data; identifying intervals in the electrogram data between adjacent R peak values; determining an average for the intervals; identifying a portion of the intervals that are at least a threshold value above or below the average; and removing the portion of the intervals from the electrogram data to generate the filtered electrogram data.

The filtering can include performing filtering based on time-domain analysis of the electrogram data, frequency domain analysis of the electrogram data, or both. The filtering can include determining one or more of ratios, products, sums, differences, weighted derivations, and integrals of two or more cardiac electrogram measures.

The vector for the electrogram data can include a PQRST complex electrogram data vector or any component thereof. The threshold value can be a threshold percentile above or below the average. The average for the intervals can be determined from only a portion of the electrogram data that is identified within a window of time from the electrogram data.

The filtering can include a second filtering process that includes identifying R peak values for R-waves in the electrogram data; determining an average R peak value from the identified R peak values; identifying a portion of the R-waves with R peak values that are at least a threshold value above or below the average R peak value; and removing the portion of the R-waves from the electrogram data to generate the filtered electrogram data.

The filtering can include removal of baseline wander, such as through use of a high pass filter. In some implementations, T-P intervals may be recognized to create a spline of the wander, which can then be subtracted to create a zero-level baseline signal.

The filtering can include using a notch filter to extract line interference and harmonics. The notch filter can be configured to operate in the 50-60 Hz frequency range, such as a 50 Hz notch filter, a 60 Hz notch filter, or a combination of these. The frequency of the notch filter can be selected automatically (e.g., a 50 Hz filter or a 60 Hz filter) based on location information that is usable to determine which line frequency is used in a particular geographic region, such as location information that is received from user input or obtained from global positioning system (GPS) data.

The filtering (or other processing of the electrogram data) can include performing respiratory compensation on the electrogram data so as to account for the patient's breathing cycle. For example, the electrogram data may be refined based on the patient's respiratory phase, whether inspiration, expiration, both, or segments thereof. The refinements may include gating, so that signals are only acquired during selected segments of the respiratory cycle and/or only during selectable respiratory rates. The refinements may include mathematical compensation for the preturbations caused by respiration to the recorded electrogram. The respiratory cycle information itself may be determined by additional sensors or measurements, or may be extracted from the ECG signal by demodulating its amplitude variations or using other techniques.

The vector for the electrogram data can include a PQRST complex electrogram data vector or any component thereof. The threshold value can be a threshold percentile above or below the average R peak value. The average R peak value can be determined from only a portion of the electrogram data that is identified within a window of time from the electrogram data. The filtering can include a third filtering process that includes identifying a vector for the electrogram data; identifying an average ECG vector; determining a statistical covariance between the average ECG vector and the vector for the electrogram data; determining one or more correlation coefficients for the electrogram data based on determined statistical covariance; and removing portions of the electrogram data with corresponding correlation coefficients that are less than a threshold correlation value to generate the filtered electrogram data.

The vector for the electrogram data can include a PQRST complex electrogram data vector.

The filtering can include a fourth filtering process that includes, for a particular P wave in the electrogram data, identifying at least a threshold number of preceding P waves; determining a mean voltage level for the preceding P waves; adjusting the elevation of the particular P wave and portions of the electrogram data surrounding or to the left of the P wave based on the mean voltage level to generate the filtered electrogram data. This process can be applied to any component of the ECG (including PQRST complex)

The filtering can include a fifth filtering process including averaging (including weighted averaging) electrogram data from the one or more leads to generate the filtered electrogram data.

The one or more waveform features can be identified from the electrogram data includes a P-wave that precedes an R-wave in the electrogram data. The P-wave includes one or more of i) a P-wave area value comprising an area underneath the P-wave and ii) a P-wave amplitude value comprising an amplitude of the P-wave.

The one or more waveform features identified from the electrogram data can include a QRS complex that comprises Q, R, and S peak points for a Q-wave, an R-wave, and an S-wave. The QRS complex includes one or more of i) a QRS area value comprising an area of a triangle formed by the Q, R, and S peak points and ii) a QRS area changes value comprising a change in the QRS area value between one or more R-waves.

Identification of the QRS complex from the electrogram data can include identifying the R peak point for the R-wave in the electrogram data; identifying the S peak point for the S-wave and the Q-wave nadir for the Q-wave based on a comparison of a first order derivative of the electrogram data to a statistically defined threshold value. The one or more waveform features identified from the electrogram data can include a T-wave that proceeds after an R-wave in the electrogram data.

The T-wave can be divided into sections based on a relationship between i) a peak of the T-wave and ii) a beginning and an end of the T-wave. The T-wave can include one or more of i) a T-wave area value comprising an area underneath the T-wave, ii) a T-wave amplitude value comprising an amplitude of the T-wave, iii) a T-wave left slope value comprising a slope value for a left portion of the T-wave, iv) a T-wave right slope value comprising a slope value for a right portion of the T-wave, and v) a T-wave center of gravity value comprising a center point under a curve of the T-wave.

The T-wave can be divided into sections such as to identify leading and trailing T-wave slopes, and the following features can be determined for each of the sections: the T-wave area value, the T-wave amplitude, the T-wave left slope value, the T-wave right slope value, and the T-wave center of gravity. Determination of one or more of the T-wave right slope value and the T-wave left slope value can include: identifying a start and end point of the T-wave from the electrogram data; identifying an inflection point at which a second derivative for a curve of the T-wave changes signs; determine i) a left point that is a threshold number of samples left of the inflection point along the curve of the T-wave and ii) a right point that is a threshold number of samples right of the inflection point along the curve of the T-wave; and determine a slope between the left point and the right point.

Determination of one or more of the T-wave right slope value and the T-wave left slope can include identifying a start and end point of the T-wave from the electrogram data; determining a first derivative between a peak of the T-wave and the end point of the T-wave; and determining a mean of a plurality of slope value samples that are derived from sample points along the first derivative. Determination of one or more of the T-wave right slope value and the T-wave left slope can include identifying a start and end point of the T-wave from the electrogram data; determining a first derivative between a peak of the T-wave and the end point of the T-wave; determining a plurality of mean slope values, wherein each mean slope value comprises a mean of a plurality of slope values for sample points along the a curve of the T-wave, the slope values being derived from the first derivative; and identifying a minimum of the plurality of mean slope values. These slopes can also be determined by any means known in the art.

Identification of the T-wave from the electrogram data can include: selecting a size for a sliding window; iteratively moving a position of the sliding window forward in time along the electrogram data and, at each iteration, determining an area under a curve defined by the electrogram data; and identifying starting and ending points for the T-wave based on positions of the sliding window when the sliding window is on a maximum area value and a minimum area value was determined. Identification of the T-wave from the electrogram data can include determining a line from a T-wave peak point to a heart rate adjusted point forward in time; evaluating vertical distances between the line and a waveform defined by the electrogram data; and identifying a point in time on the waveform with a maximum vertical distance as the start or end point of the T-wave. The T-wave can also be determined by any means known in the art.

Determining of the one or more estimated analyte levels can include determining a virtual lead (i.e. a lead that is determined by performing one or more operations on measured electrical data) that indicates the one or more estimated analyte levels for the patient based on the electrogram data derived from the one or more leads that sense physiological electrical activity of the patient. Identifying the one or more correlations between values of the one or more waveform features and analyte levels can include transforming a data matrix representing the electrogram data for the one or more leads into a virtual lead space that indicates the one or more estimated analyte levels for the patient, the transformation of the data matrix generating one or more virtual leads that indicate analyte levels for the patient; and statistically analyzing the one or more virtual leads to identify the one or more correlations. Virtual leads can also be created using PCA or ICA (independent component analysis).

The transforming of any of the leads (virtual or not) can include principal component analysis (PCA) or ICA for the data matrix. The transforming can include PCA or ICA of the data matrix and unsupervised optimal fuzzy clustering (or any other clustering method) of a coefficient matrix generated from the PCA or ICA of the data matrix. The statistically analyzing can include performing multiple linear regression or multivariate regression analyses on the one or more virtual leads. The analyte levels can be selected from the group consisting of: potassium, calcium, magnesium, phosphorous, and anti-arrhythmic drugs.

The output information can identify one or more ranges that are associated with the one or more estimated analyte levels. The output information can identify whether the one or more estimated analyte levels fall within one or more ranges. The output information can identify at least a portion of the one or more estimated analyte levels. In addition, the output information can be used to specifically estimate an analyte, or to detect a change in an analyte level (with or without specifying an absolute value).

The method can further include recording, based on electrogram data and corresponding analyte level measurements, the one or more correlations that are personalized to the specific patient or universal to a population. The method can further include generating a mathematically characterized template that is specific to the patient or universal to a population and that provides a baseline of analyte levels for the patient; and comparing the one or more estimated analyte levels for the patient to the template to identify deviations from the template. Both the universal template for a population and the personalized template for each individual patient can be learned by supervised and unsupervised machine learning classification and clustering techniques.

The method can further include performing time domain and/or frequency domain analysis with regard to the electrogram data.

The method can further include performing a wavelet transform with regard to the electrogram data. The method can further include modeling the electrogram data using a hidden Markov model. The method can further include performing linear discriminate analysis with regard to each characteristic of the electrogram data. The electrogram data can be obtained from an implanted recording system.

The implanted recording system can include a dedicated system for assessing analyte levels. The implanted recording system can include an implantable loop recorder that is capable of being used to diagnose arrhythmia or syncope. The implanted recording system can be included in a pacemaker, defibrillation, or resynchronization system. The implanted recording system can include an indwelling dialysis catheter. The implanted recording system can include an implant. The implant can be an abdominal implant, a central nervous system implant, or a vascular implant. The implanted recording system can include an ingestable device. The ingestable device can include an electronic capsule or tablet.

The method can further include determining, based on the electrogram data, a risk that the patient will develop ventricular arrhythmias. The method can further include determining, based on the electrogram data, a risk that the patient will develop atrial fibrillation. The method can further include determining, based on the electrogram data, a risk that the patient will experience drug-induced proarrhythmia. The computer system can include a smartphone, a tablet computing device, a notebook computer, or cloud-based analysis.

In some implementations, a computer-implemented method can include accessing, by a computer system, electrical signal data for a patient, wherein the electrical signal data is obtained using one or more leads that sense physiological electrical activity of the patient; identifying, by the computer system, one or more waveform features from the electrical signal data; identifying, by the computer system, one or more correlations between values of the one or more waveform features and analyte levels; determining, by the computer system, one or more estimated analyte levels in the patient based on 1) the one or more waveform features identified from the electrical signal data and 2) the one or more correlations; and outputting, by the computer system, information related to the one or more estimated analyte levels.

The electrical signal data can be selected from a group consisting of electrocardiogram (ECG) data, electroencephalography (EEG) data, EMG data (see previous comment) and data that characterizes the patient's response to a localized stimulation. The method can further include determining information that characterizes the patient's body position or breathing profile at a time when the electrogram data is obtained. Determining the information that characterizes the patient's body position or breathing profile can include processing signals obtained from an accelerometer connected or otherwise coupled to the patient. The one or more waveform features can be identified in response to determining that the patient's body position matches a predetermined body position or portion of the respiratory phase.

The method can further include determining that the patient's body position or respiratory phase at the time when the electrogram data is obtained has changed from a predetermined body position or respiratory phase, and in response to determining that the patient's body position or respiratory phase has changed from the predetermined body position or respiratory phase, adjusting the one or more estimated analyte levels.

The method can further include monitoring the patient's heart rate; and determining that the patient's heart rate is within an acceptable range of a baseline heart rate, wherein the electrogram data is accessed in response to determining that the patient's heart rate is within the acceptable range. The acceptable range can be ten beats per minute above or below the baseline heart rate. Multiple bins of heart rates could be obtained across the range of the patient's rates.

The method can further include determining that the patient's heart rate at a time when the electrogram data is obtained deviates from a baseline heart rate, and in response to determining that the patient's heart rate deviates from the baseline heart rate, adjusting the one or more estimated analyte levels.

The window of time can be defined by at least one of a start time and an end time, the start time and end time corresponding to a particular time of day. The window of time can be determined based on a time when the patient's body position or heart rate matches a baseline body position or a baseline heart rate.

Determining the virtual lead that indicates the one or more estimated analyte levels for the patient can include determining a difference between adjacent unipolar electrodes in the one or more leads and comparing the difference to a signal from a local bipole.

The method can further include determining a time-based derivative of the electrogram data, wherein the one or more waveform features are identified from the time-based derivative of the electrogram data. The method can further include generating, based on a determination that the one or more estimated analyte levels for the patient deviate at least a threshold amount from baseline analyte levels in the patient-specific template, an alert to notify a user of the deviation. Generating the mathematically characterized personalized template can include drawing blood from the patient and measuring one or more components to determine the baseline of analyte levels.

A personalized template can be developed for individual patients, such as by supervised machine learning techniques, unsupervised machine learning techniques, and/or clustering techniques. In some implementations, individual patient templates can be initially generated based on population data from other patients to initially seed the template.

In some implementations, a binning technique can be employed in which the electogram data generally includes only data that has been obtained when the patient is in a pre-defined condition. The pre-defined condition may relate to the patient's heart rate, body position, or other conditions. For example, the electrogram data may include only data that has been acquired when the patient's heart rate is within an acceptable range of a baseline heart rate, or the electrogram data may include only data that has been acquired when the patient is in a particular body position (e.g., supine or standing). Condition-specific templates may be developed for patients in some implementations. For example, different templates may apply depending on whether the patient is standing or sitting, and/or depending on a range that the patient's heart rate is within when the electrogram data is acquired. In some implementations, a common template may apply across a range of conditions, but compensations may be mathematically performed on the electrogram data to account for varying conditions of the patient, such as if the electrogram data was acquired while the patient's heart rate was outside of an acceptable range.

Determining the risk that the patient will develop ventricular arrhythmias can include determining a center of gravity or a T-wave slope based on the patient's electrogram data.

The electrogram data can include one or more of electrocardiogram data, brain electrogram data, muscular electrogram data, myoelectrogram data, and neuro-electrogram data.

The one or more leads that sense physiological electrical activity of the patient can be physically attached to the patient, or can be not physically attached to the patient.

The details of one or more implementations are set forth in the accompanying drawings and the description below. Various advantages can be provided by certain implementations. For example, improved accuracy of ECG data-based quantification of the concentration of potassium, calcium, magnesium, phosphorous, and anti-arrhythmic drugs in the blood can be obtained. For instance, the disclosed techniques can enable a prediction accuracy level of above 70%, and above 90% in some instances. In another example, accuracy can be improved based on using the values of the parameters involving the T wave. In some examples, additional advantages may be realized, including, for instance, permitting near real-time ambulatory assessment of analytes without the need for blood tests, permitting continuous screening of the ECG to identify changes using compressed signals, and conserving computing device power, such as battery power in mobile applications. In one example, the disclosed techniques permit risk stratification for the development of atrial or ventricular arrhythmias in near real-time in ambulatory individuals. None, some, or all of the advantages may be realized in various implementations of the disclosed techniques.

Other features, objects, and advantages of the invention will be apparent from the description and drawings, and from the claims.

### DESCRIPTION OF DRAWINGS

FIG. 1 depicts example lead positioning on a patient.

FIG. 2 is a graph that depicts shows observations of R-R intervals.

FIG. 3 is a graph that depicts R peaks that are dropped from the ECG observations.

FIG. 4 is a graph that depicts a plot of ECG heart beats showing p-elevation correction.

FIG. 5A is a graph that depicts an example of 15 minutes of data after the averaging stage.

FIG. 5B depicts five example graphs that depict ECG data after application of one or more of the filtering stages discussed in this document.

FIG. 6 depicts time domain ECG features.

FIG. 7 is a graph that depicts the calculation results of center of gravity of the T-wave.

FIG. 8 is a graph that depicts QRS complex detection.

FIGS. 9A-B depicts detection of a T-wave with a sliding window technique that is based on the assumptions of T-wave concavity, and on QRS-complex detection.

FIG. 10 depicts detection of a T-wave through a second example technique.

FIG. 11 depicts smoothing with a low pass filter.

FIG. 12 is a graph that depicts a first example technique for T-wave slope calculations.

FIG. 13 is a graph that depicts a second example technique for T-wave slope calculations.

FIG. 14 depicts the results of linear regression analysis indicating a relationship between the blood potassium level and the shapes (PQRST complexes) in the ECG signal.

FIG. 15 is a block diagram of example computing devices.

Like reference symbols in the various drawings indicate like elements.

### DETAILED DESCRIPTION

This document describes computer-based techniques for quantifying the concentration of analytes, such as potassium, in a patient's blood based on physiological electrical data (electrogram data). The physiological electrical data may be obtained using any suitable technique such as electrocardiogram (“ECG”) measurements (which may include surface, intracardiac, or subcutaneous ECGs, or measurements obtained using a pacemaker implanted in a patient's body, or defibrillators, for example). Other physiological electrograms may also be employed, including brain electrograms (“EEG”), muscular electrograms, myoelectrograms that cover smooth and striated muscle, for example, and neuro-electrograms. Either or both tonic and resting physiologic electrograms may be employed, as well as electrograms that measure responses to provocations such as evoked stimuli or extrinsic electrical stimulation or other stimulation.

In the context of this document, electrogram data generally refers to an electrical recording of any electrically active biological tissue, whether recorded from a traditional surface ECG electrode, custom body surface electrodes that may vary in size, shape, and inter-electrode distance, for example, or from intracoporeal electrodes, whether they be subcutaneous, intracardiac, or within other tissues or natural cavities. Electrograms from which such data is obtained may be spontaneous, or in response to a stimulus or provocation, and may be recorded from contact or non-contact electrodes. By way of example, the electrogram data may be obtained from one or more physiological electrograms including electrocardiograms (ECG), brain electrograms (EEG), muscular electrograms, myoelectrograms, and neuro-electrograms.

While the term “computer-based” is applied, it is recognized that this may refer to any suitable form of computer processing, including mobile-based processing. For example, the techniques disclosed herein may be implemented at least in part by a mobile computing device such as a smartphone, tablet, or notebook computer that communicates with a system of wearable electrodes. These techniques may also be implemented in wearable ECG patches or implantable devices. These techniques permit data compression and distribution of processing among various aspects of such a system, to enable near real-time, frequent, analyte assessment in ambulatory/outpatient individuals. This may be particularly useful in dialysis patients who are at risk for abnormal analyte levels (e.g., hyperkalemia), patients with cardiac disease, and/or renal insufficiency. This document discusses quantifying concentrations of potassium in some examples, although similar techniques may also be used to quantify concentrations of other analytes as well, including quantification of drug levels. Additionally, this paper broadly uses the term “patient” to generally include any person from whom electrogram data is obtained, regardless of their clinical status for example.

This document describes the results of two studies that were used to develop these techniques: one of human subjects, and one of animals. The human study includes 12 patients under hemodialysis. The animal study is based on analysis from 5 pigs. The described techniques use three general stages: (1) Pre-Processing, e.g. filtering, (2) Pattern Recognition and Decomposition, accomplished by means of principal component analysis (“PCA”) and ECG characteristics, Pattern Classification by means of Unsupervised Optimal Fuzzy Clustering using PCA and ECG characteristics, and (3) Potassium evaluation using linear regression on ECG parameters and PCA coefficients.

Regarding pre-processing, noise reduction was the first and foremost initial process to be performed, so that a smooth signal may be obtained. The following description describes the test process, the filtering processes used to get smooth and reliable ECG signals and the classification and potassium evaluation methods and results. The outcomes of this stage allow a determination of approximate potassium levels by analyzing the filtered data, comparing it to the potassium levels measured from drawn blood.

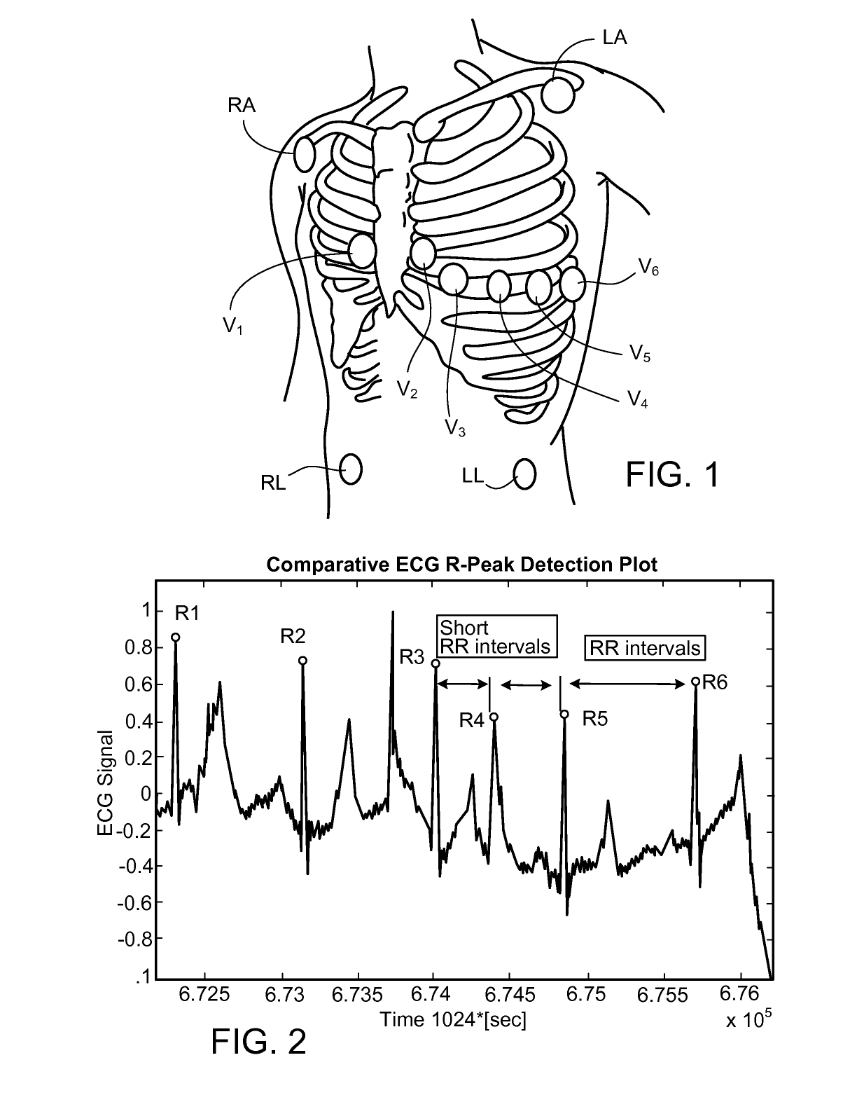

Data used in the human study was obtained as discrete ECG data of 12 patients from a Siesta 802 monitoring system. The Siesta 802 monitoring system is just one example of a system that can be adapted for the purposes described herein. The signal was sampled at 1024 bps, although those skilled in the art will recognize that other sampling rates may also be used. The ECG samples were taken from 9 Leads (RA, LA, LL, V1, V2, V3, V4, V5 and V6 as depicted in FIG. 1) which were transformed to standard 12 Leads (I, II, III, aVL, aVR, aVF, V1, V2, V3, V4, V5 and V6). Other arrangements of lead positions may also be used, and various subsets of the standard 12-lead configuration may also be used in some implementations. Blood draws were taken from the patients while under hemodialysis process, observing Potassium levels, as well as the levels of other electrolytes. The tested information was taken from consecutive dialysis patients, since they have wide fluctuations in serum potassium. While the example study described herein obtained ECG samples from a 9-lead system, generally ECG samples can be collected from any number of leads, including 1 or 2 leads to collect data used to assess analyte levels. Similarly, electrical data signals other than ECG may also be collected such as, for example, subcutaneous ECG data, intracorporeal electrodes in any body cavity or chamber, electroencephalography (EEG) data samples and data samples in response to various stimuli applied to the patient.

The test was performed in 3 segments, each 15 minutes long, starting from 0 m as the baseline, increasing, in the following segments, to 90 m and 180 m. The potassium level in the blood samples and the ECG data were recorded, the ECG signal was then analyzed using signal processing tools in order to evaluate the potassium level, while using the potassium values taken from the blood samples as references. This process was repeated for each of the segments. The test may also be performed according other parameters. For example, the segments may be shorter or longer than fifteen minutes, and the number of segments may also vary.

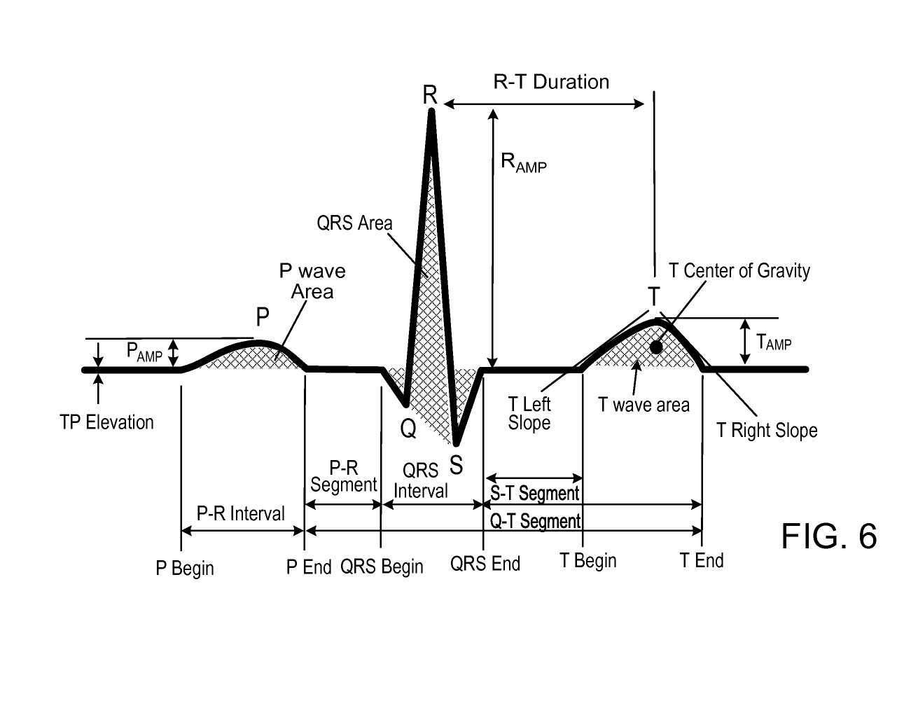

Regarding filtering the obtained ECG data, the data signal was obtained from the ECG monitoring system's own Analog to Digital transformer. Analysis of the data was performed programmatically in a numerical computing environment (Matlab). The process starts with finding the R peak points; once the R peaks are determined, all other waves (P, Q, R, S and T as depicted in FIG. 6) may be identified, and the patient's heart rate may be calculated. The ongoing ECG signal was divided into small segments, observations, each holding sampled ECG data corresponding to one blood cycle passing through the heart (one heartbeat). All small segments (N length˜800 ECG samples, depends on the average Heart Rate of the patient) were stored in 15 database matrices (length N×M, where M˜70 is 1 minutes ECG data). The 15 matrices together hold 15 minutes of ECG data. The small segments were adjusted to the R point in the time axis.

A plurality of filtering stages can be used, alone or in any of a variety of possible combinations. In a first filtering stage (heart rate filtering), the ECG observations that fell outside the range of 25% above and 25% below the 15 minutes average R-R interval are dropped. Referring to FIG. 2, which shows observations of R-R intervals, ECG observations including R3, R4 and R5 were dropped from the database matrices. Other suitable ranges, more or less than the +/−25% range may also be used. Thus, outlying R-R intervals that are exceedingly long or short may be excluded from the analysis.

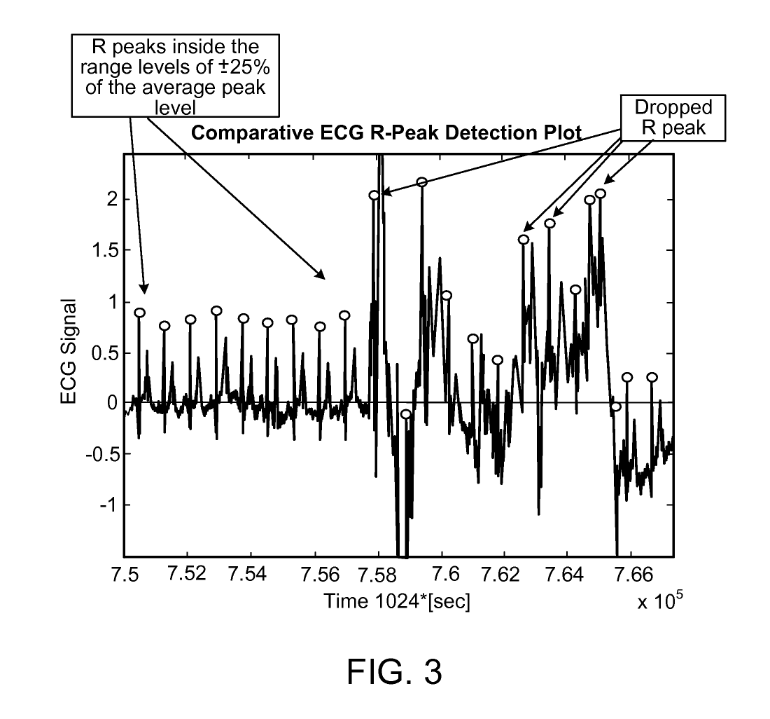

In a second filtering stage (R peak level filtering), ECG observations with peaks that fell outside the range of 25% above and 25% below the 15 minutes average R-Waves are dropped. FIG. 3 depicts several such peaks that are dropped from the ECG observations. For instance, the ECG observations in the right side of the plot depicted in FIG. 3 include high level R waves were dropped from the database matrices

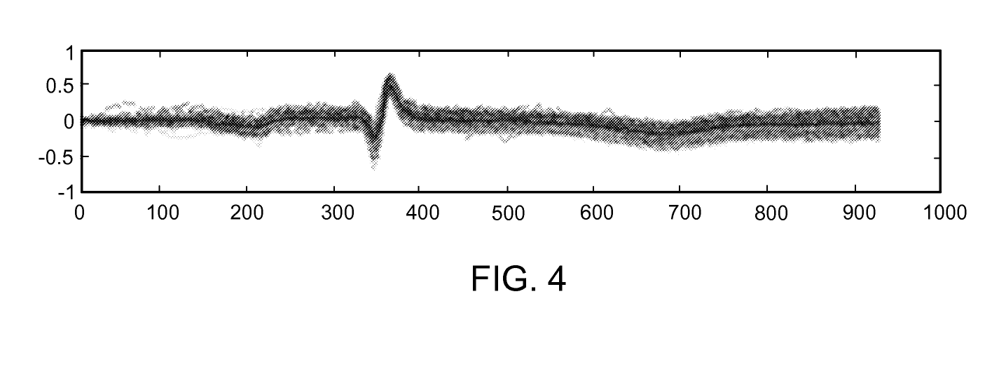

In a third filtering stage (correlation to the average filtering), ECG observations whose correlation to the average ECG is below 90% are dropped. FIG. 4 shows such an observation, denoted in green, while the average ECG is denoted in red. For instance, the ECG Observation denoted in green with less than 90%, correlated to the averaged ECG denoted in red. This correlation filter can rely on statistical covariance, the measure of how much two random vectors change together. For instance, the covariance between two (m×1) dimensional vectors X (ECG average vector) and Y (individual PQRST complex ECG data vector) is equal to:

COV(X,Y)=E[(X−E[X])(Y−E[Y])^{T}]

where: E[X] and E[Y] are the means of X and Y respectively; (Y−E[Y])^{T} is the transposition of the vertical vector (x−E[X]); the covariance matrix dimension is (m×m); the (i,j)-th element of this matrix is equal to the covariance between the i-th scalar component of X and the j-th scalar component of Y. Correlation can simply be understood as a normalized version of covariance, called correlation coefficient. The correlation coefficient between the vector of means and each data vector can be equal to:

$${ρ}_{X , Y} = \frac{COV ( X , Y )}{\sqrt{{( {σ}_{X} )}^{2} {( {σ}_{Y} )}^{2}}}$$

where: ρ_{X,Y} is the correlation coefficient matrix (2×2 dimension); COV is the covariance matrix; and (σ_{X})² and (σ_{Y})² are the variances of X and Y respectively. The magnitude of the correlation coefficient shows the strength of the linear relation between the two vectors. Vectors whose covariance is zero can therefore be uncorrelated.

To recap, this filtering stage (correlation to the average filtering) involves dropping ECG observations whose correlation with the mean, as represented by their correlation coefficient with the average ECG is less than 90%.

In a fourth stage of filtering (baseline wandering correction), the baseline wandering of the ECG signal can be corrected such that the P-elevation along with the entire ECG heart beat segment can be adjusted to 0. An example of such filtering is depicted in FIG. 4, which is a graph that shows the red plot being adjusted to the 0 DC level on the left side of the P wave. This filtering is accomplished by finding the mean level of threshold number of samples (e.g., 20 samples) interval prior to the P wave (the values between 350-370 ms in FIG. 4), and vertically shifting the entire ECG heart beat sample by that value. In some implementations, baseline wondering correction can be performed by applying spline-based correction to the ECG signal, by applying a frequency filter such as a high-pass, low-pass, or band-pass frequency filter to the ECG signal, or other manners of restoring the isoelectric line (P-elevation) to a zero level.

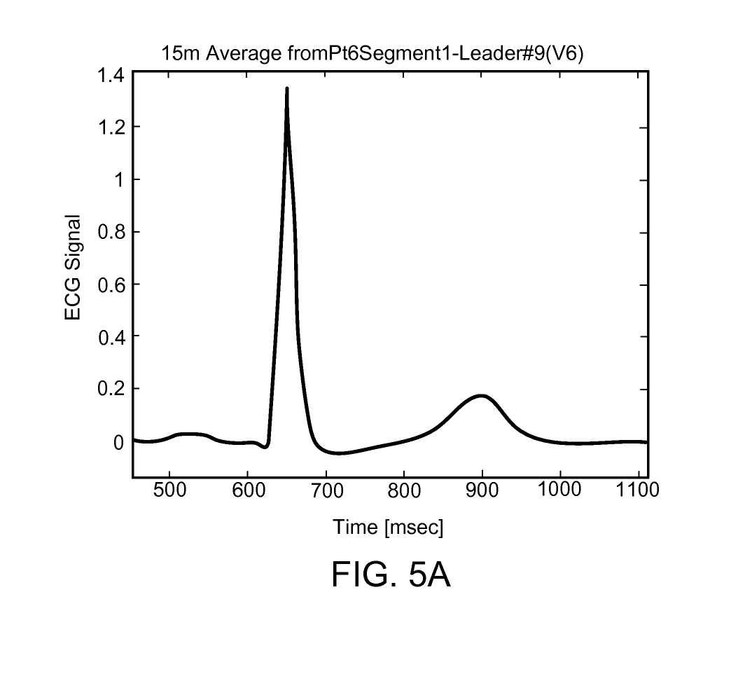

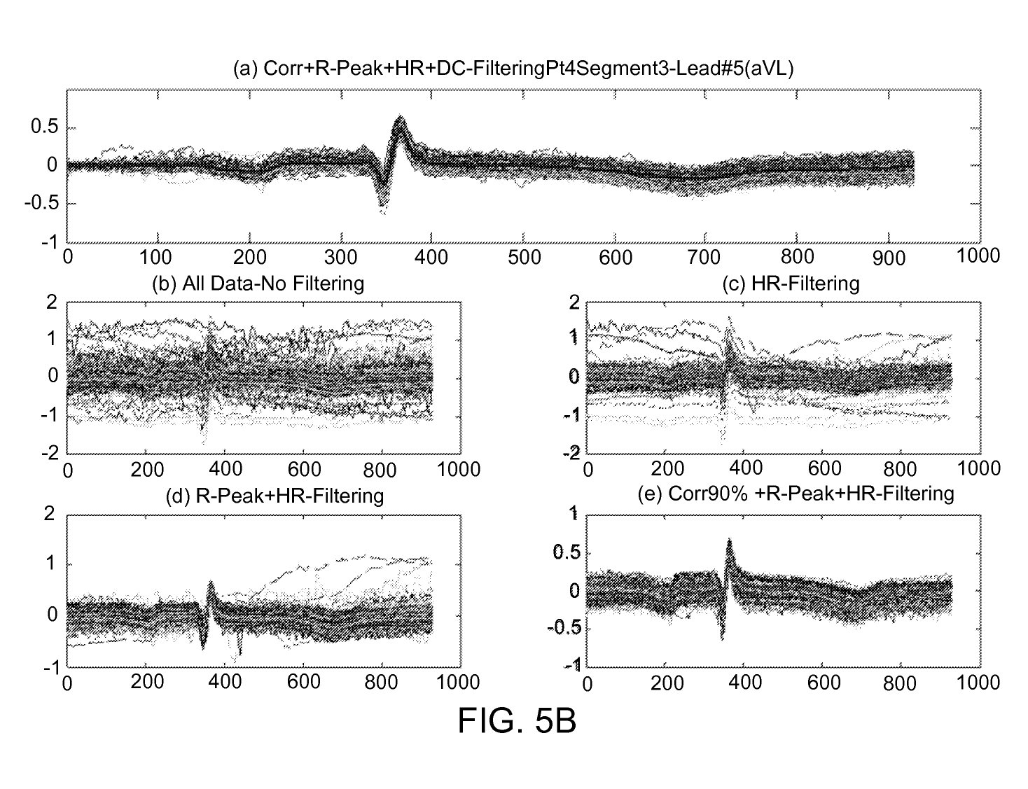

In a fifth filtering stage (averaging), the pre-processing after removing the unwanted components is averaging the remaining ECG complex for each one minute in the segment. The averaging process can be performed in all segments (e.g., 3 segments) and for all leads (e.g., 12 leads). For instance, as depicted in FIG. 5A below, an example of 15 minutes of data after the averaging stage is depicted.

The pre-processing filters described above can remove distortions which may interrupt the analysis, but in the other hand there is a risk that the dropped ECG components may include also important information about the potassium level in the blood. Spatially, when removing uncorrelated components to the 15 minutes averaged ECG, it is assumed that the averaged ECG is a desired end result for the process. In practice, the entire filtering process may drop about 15% of the ECG components and it can be assumed that this has a minor impact on the results. Following the pre-processing, a basic data set generated and arranged in 12 matrices, with each matrix representing an ECG lead, with 45 ECG averages of one minute, can be generated. Each 15 minute average is associated with a potassium level measured from drawn blood. These matrices can be used in the clustering process and the potassium evaluation analysis. FIG. 5B depicts five example graphs that depict ECG data after application of one or more of the filtering stages discussed in this document.

Research has indicated that a potassium change in the blood has a great effect on the potential of myocytes (heart cells). By measuring myocyte potentials using ECG techniques, analyte levels, such as potassium, in a patient's blood can be determined. In the studies discussed in this document, several ECG characteristics were tested, and a quantification method of potassium based on P-wave, QRS complex and T-wave was developed. This study also tests a new method to quantify potassium from T-wave Center of Gravity and the results shows high correlation to serum potassium level.

To systematically subject these changes to predictive statistical analysis (linear regression and clustering), the ECG features were extracted as shown in FIG. 6. These features included: T wave area, T wave area changes, T wave amplitude, R wave amplitude, QT-interval, QT/(RR)̂0.5 (Bazett's formula), QRS area, QRS area changes, T Right slope, T wave Right slope/T wave Area, T wave Right slope/T wave Amplitude, T Left slope, T wave Left slope/T wave Area, T wave Left slope/T wave Amplitude, T wave amplitude/R wave amplitude, T wave Area/R wave Area, P wave amplitude, P wave area and a new feature T-wave Center of gravity.

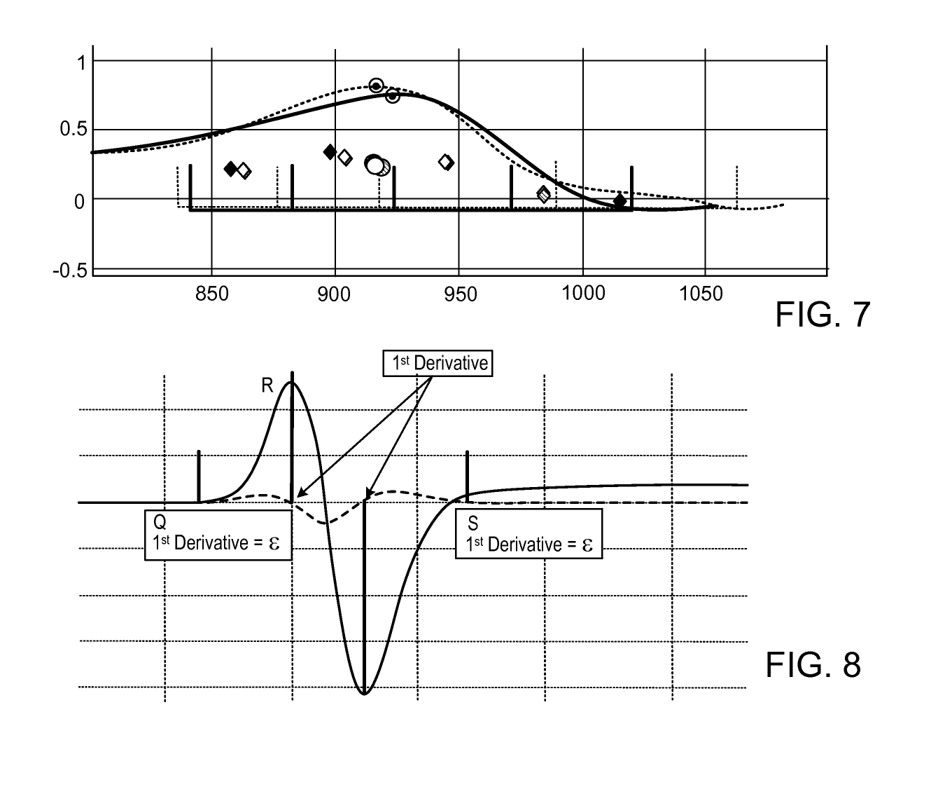

FIG. 7 is a graph that depicts the calculation results of center of gravity of three T wave segments (in red, green and blue circles), and a center of gravity calculation of four quarters of the T wave marked (in red, green and blue diamonds). Automated edges detection was implemented (see edges detection methods section).

Linear regression between each feature and the potassium performed in two dimensions, and a linear line was estimated to extract potassium level from the feature. The center of gravity (COG) feature, in the other hand, can be three dimensional: time value of center of gravity, ECG level value (e.g., voltage amplitude) of center of gravity, and potassium level. The Human study included three potassium measures which only together with the COG defines 3 point in three dimensional spaces. For parameters that have good results in the linear regression, unsupervised optimal fuzzy clustering (UOFC) can be performed (sometimes in combination with PCA) on those parameters to determine whether there have been any relevant changes in potassium values. PCA on ECG waveform analysis can be performed to derive waveform coefficients. Linear regression of those coefficients can also be used to identify changes in potassium levels. PCA permits compressed signals to represent the waveform, and UOFC identified a change in the waveform when potassium values change by 0.2 mEq/L.

The feature T-wave center of gravity was projected twice, once to the time dimension and secondly to the ECG level; the new features now are, T-wave Center of gravity (time depended), T-wave Center of gravity (amplitude depended).

The QRS complex can be detected in any of a variety of appropriate ways. For example, referring to FIG. 8, the QRS detection can begin with R peak detection (e.g., detection technique developed by Sergey Chernenko and as indicated on http://www.librow.com). The Q and S waves can be detected by comparing the 1^{st} order derivative of the ECG to a statistically defined threshold E. To detect the part of the area in the T wave which is most correlated to the potassium level, the T wave was vertically divided into four parts, as depicted in FIG. 8, to be statistically analyzed.

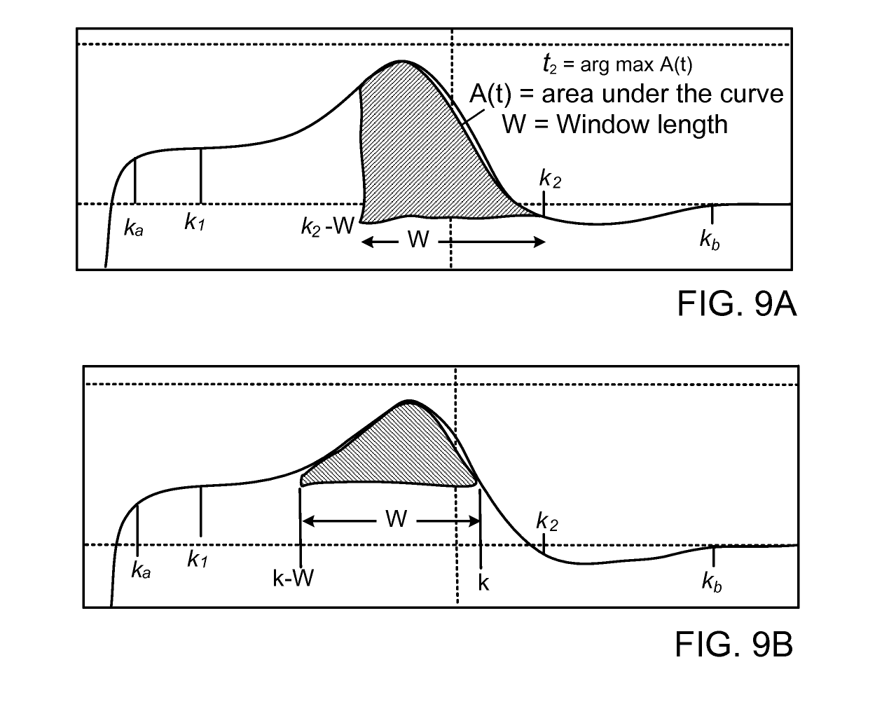

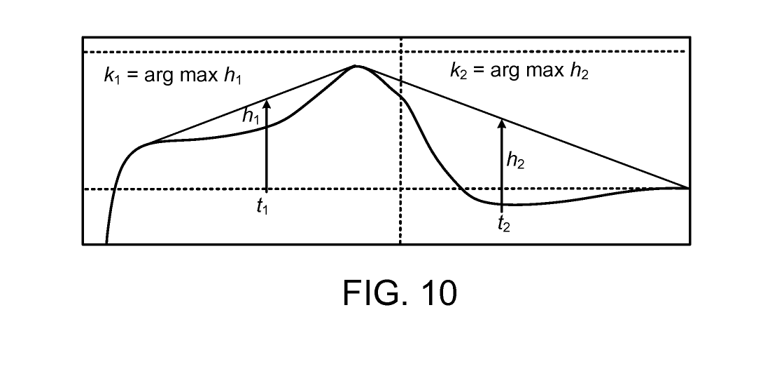

A variety of techniques can be used to calculate the values of features from the ECG, edges of the P-wave, the QRS complex, and the T-wave. For example, the techniques that are depicted in FIGS. 9A-B and 10 can be used to detect such features.

FIGS. 9A-B depict detection of the end point of a T-wave with a sliding window technique that is based on the assumptions of T-wave concavity, and on QRS-complex detection. For this technique, let sₖ k=1, 2 . . . n be the k^{th} averaged cardiac cycle of ECG signal value, where n is the number of samples in the averaged cardiac cycle. For each averaged cardiac cycle, an interval [kₐ,k_{b}] is roughly delimited so that the T-wave end is inside this interval, and the end of the average is far enough to include the T end. Let the following equation define the area of the sliding window (size w) under the T-wave:

$${A}_{k} = {\sum }_{j = k - w + 1}^{k} ( {S}_{j} - {S}_{k} )$$

In order to reduce of the effect of measurement noise, in the above formula S̅ₖ should be used instead of Sₖ, where S̅ₖ is the mean value of the signal in a small window around k. Then for each instant k between kₐ and k_{b}, the value of Aₖ is computed and the T-wave end is located at the value of k maximizing or minimizing Aₖ, as summarized in the following pseudo-code for the technique:
1. Choose the sliding window size w and the smoothing window size p<<w.
2. Choose also a threshold λ>1 for T-wave morphology classification.
3. Read one averaged cardiac cycle of the ECG
4. Choose the values of kₐ and k_{b} between R peak and the end of the ECG cycle to confine the T-wave end search.
5. For each instant k=kₐ, kₐ+1, . . . , k_{b} compute S̅ₖ and Aₖ.

$${k}_{2} = arg {max}_{k ∈ [ {k}^{′} , {k}^{″} ]} \vert {A}_{k} \vert$$

6. Repeat from step 1 to find k₁

FIG. 10 depicts detection of the end point of a T-wave through a second example technique. As part of this second example technique, a line is drawn from the top of the T wave to a heart rate-adjusted point forward in time. The vertical distance from each sample point on the waveform to the line is computed, and the time point of the maximum vertical distance is considered the T-wave offset.

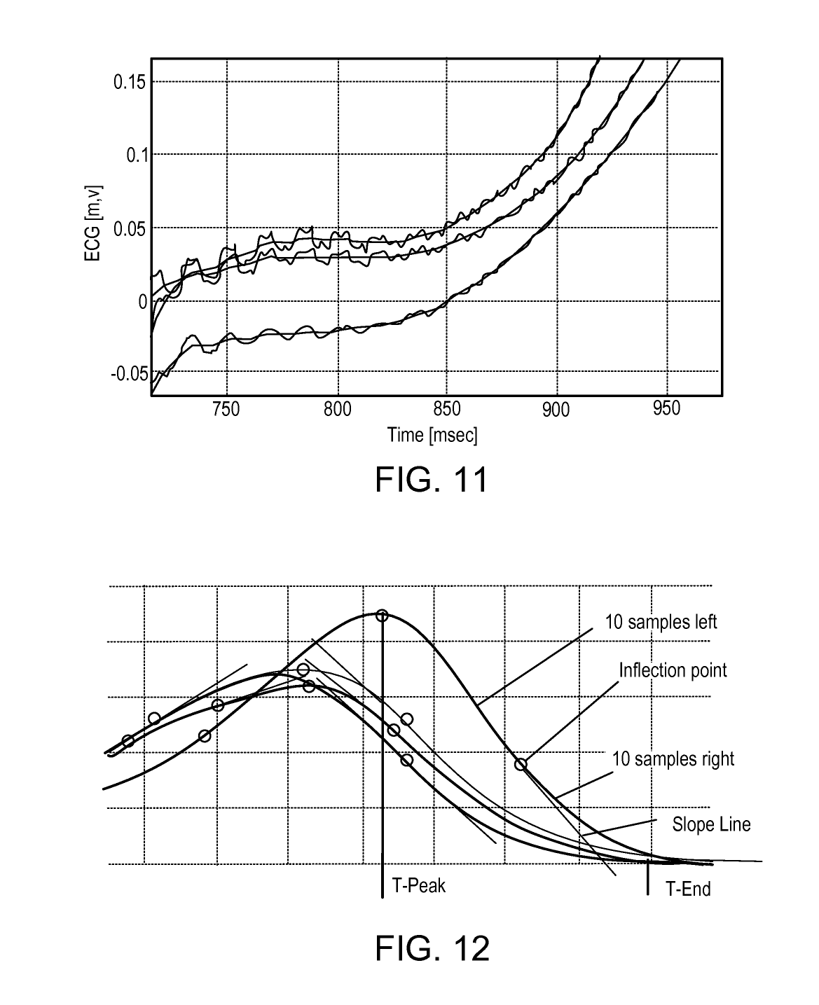

The averaging process of 15 minutes removes most of the artifacts in the measured ECG signal; however, another low pass filter is implemented for cases where the averaging process only didn't provide a good smoothed ECG signal. Referring to FIG. 11, which depicts smoothing with a low pass filter, original and smoothed (low pass filter) comparison of 3 segments of 15 minutes Averaged ECG. The black line which is the filtered signal shows reduction of 60 Hz. Since the calculation of slope is sensitivity of the shape of the curve, if the curve is smooth then a reliable and correct slope is calculated, but if 60 Hz noise, for example, is mounted on the ECG as shown in FIG. 11 then slope calculation may indicate a wrong value. Features including the parameter T-wave slopes may be analyzed and compared with and without low-pass filter. In some implementations, features other than the T-wave slopes can be analyzed and compared with and without low-pass filter.

Research has shown that features including the parameter of T wave slopes (right and left slope) are highly correlated with the potassium concentration in blood. Four methods of T wave slope calculations were analyzed and are described below. The right slope can be calculated from T peak to end of T wave as determined in edges detection procedure. The left slope can be calculated from T peak to end of T wave as determined in edges detection procedure.

Referring to FIG. 12, which depicts a first example technique for T-wave slope calculations, an inflection point (a point on a curve at which the second derivative changes signs) can be used to generate T-wave slope calculations. The curve can change from being concave upwards (positive curvature) to concave downwards (negative curvature), or vice versa. Pseudo-code for such an example technique includes:
1. Define the T wave edges for T wave right (or left) slope calculation; choose one of the methods defined above. In this case the edges are T-peak and T-end.
2. Find the inflection point, Detect the point where the samples change sign.
3. Mark 2 points on the curve 10 samples left and 10 samples right.
4. Calculate the slope of a straight line passing between the two Points.

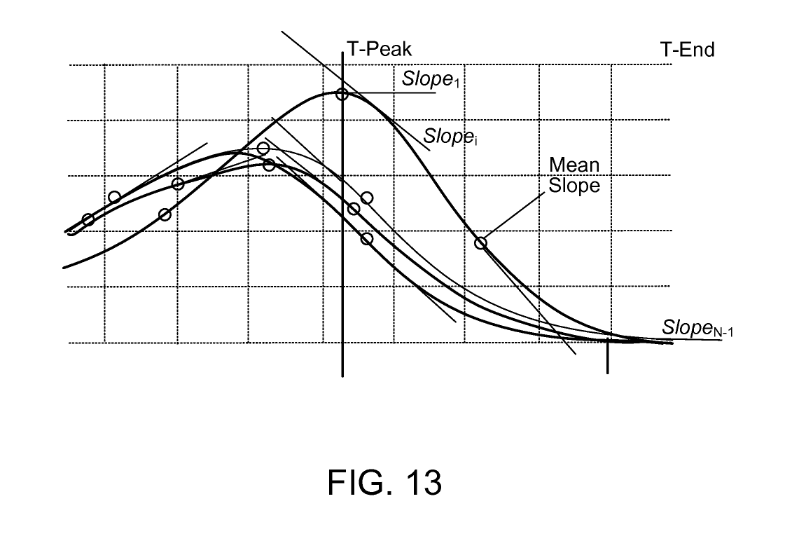

Referring to FIG. 13, which depicts a second example technique for T-wave slope calculations, mean of slopes can be used to generate T-wave slope calculations. Pseudo-code for such an example technique includes:
1. Define the T wave edges (i.e., T-wave peak and T-wave end point)
2. Calculate the 1^{st} Derivative between each two incremental samples in the interval [T-peak, and T-end].
3. Calculate the mean of the slopes.
   The following formulation can be used to implement this technique:

$$\begin{matrix}1 st {derivative}_{i} = {Slope}_{i} \\ = \frac{{S}_{i + 1} - {S}_{i}}{{Time}_{i + 1} - {Time}_{i}} ;\end{matrix}$$

$$i = 1 , 2 … N - 1$$

Where:

Sᵢ is the i^{th} ECG T wave signal value,

Timeᵢ is the ECG T wave sample number,

N is the number of samples in the ECG T wave,

$$Mean Slope = \frac{1}{N - 1} {\sum }_{i = 1}^{N - 1} {Slope}_{i}$$

In an third example technique, when the T wave is smooth a fit in the least mean sense can be used as follows:
1. Define the T wave edges
2. Calculate the 1^{st} Derivative between each two incremental samples in the interval [T-peak, and T-end].
3. Calculate the total mean slope
4. Calculate the least mean of the slopes

$$Formulation$$

$$Minimum of \{ \begin{matrix}Mean {Slope}_{1} = \frac{1}{N - 1} {\sum }_{i = 1}^{N - 1} ( {Slope}_{1} - Mean Slope ) \\ Mean {Slope}_{2} = \frac{1}{N - 1} {\sum }_{i = 1}^{N - 1} ( {Slope}_{2} - Mean Slope ) \\ ⋮ \\ Mean {Slope}_{N - 1} = \frac{1}{N - 1} {\sum }_{i = 1}^{N - 1} ( {Slope}_{N - 1} - Mean Slope )\end{matrix}$$

In a fourth example technique, if 60 Hz noise is mounted on the ECG and the T wave is not smooth, then the best fit in the least mean squared sense can be used as follows. The same as the least mean algorithm only this time use least squared mean.

$$Formulation$$

$$Minimum of \{ \begin{matrix}Mean {Slope}_{1} = \frac{1}{N - 1} {\sum }_{i = 1}^{N - 1} {( {Slope}_{1} - Mean Slope )}^{2} \\ Mean {Slope}_{2} = \frac{1}{N - 1} {\sum }_{i = 1}^{N - 1} {( {Slope}_{2} - Mean Slope )}^{2} \\ ⋮ \\ Mean {Slope}_{N - 1} = \frac{1}{N - 1} {\sum }_{i = 1}^{N - 1} {( {Slope}_{N - 1} - Mean Slope )}^{2}\end{matrix}$$

An example method was developed to determine one virtual lead which represents the 12 leads ECG signal; the algorithm uses the principal component analysis (PCA) coefficients to calculate a linear combination of 12 leads signal and generate the virtual lead. Pseudo-code for such an example method using PCA analysis in lead space is provided as follows:
1) The Data set of each 15 minutes averaged ECG segment #i containing 12 leads can be expressed in a matrix form

$${D}^{i} = [ \begin{matrix}{D}_{1}^{i} ( 1 ) & … & {D}_{12}^{i} ( 1 ) \\ ⋮ & ⋱ & ⋮ \\ {D}_{1}^{i} ( N ) & … & {D}_{12}^{i} ( N )\end{matrix} ]$$

Where:
D is the Data matrix, containing 12 columns; each represents an average of 15 minutes samples
i is the number of the segment (the human study includes 3 segments)
N number of samples in each record (lead),
12 number of records (leads)
2) Use the first segment Data for training to calculate a coefficient matrix and use it to calculate the virtual lead at each 3 segments.
3) Calculate the covariance matrix of Data segment #1 D¹ (size N×N):

cov=E{(D¹=μ_{D}_{¹})(D¹−μ_{D}_{¹})^{T}}
Where:
μ_{D}_{¹} is the averaged ECG vector of all 12 records (leads) of segment #1.

$${μ}_{{D}^{2}} = \frac{1}{12} {\sum }_{i = 1}^{12} {D}_{i}^{1} ( n ) ;$$

$$\{ n = 1 , 2 , … , N \}$$

4) Calculate eigenvalues λᵢ, (i=1, 2, . . . , N) and there corresponded N eigenvectors of the covariance matrix; they are the solution of the equation: det(G−λI)=0 (I is the identity matrix). The basis waveforms are the eigenvectors of the record set covariance matrix, which represents the correlation between all records, and they constitute an orthogonal basis of the set of records.
5) Arrange the eigenvectors in decreasing order of their eigenvalues (Large eigenvalue=Large contribution to reconstruction of all records in the set).

λ₁≧λ₂≧ . . . ≧λ_{N}
6) Ignore the zero eigenvalues and use only the L nonzero values.

λ₁≧λ₂≧ . . . ≧λ_{N}
7) Use the first L eigenvectors from the eigenvectors matrix to define a (L×N) transformation matrix whose rows are the corresponding eigenvectors.

$${G}_{L} = [ \begin{matrix}{G}_{1} ( 1 ) & … & {G}_{1} ( N ) \\ ⋮ & ⋱ & ⋮ \\ {G}_{L} ( 1 ) & … & {G}_{L} ( N )\end{matrix} ]$$

8) Compute the (L×12) coefficients matrix:

Y_{L}=G_{L}(D¹−μ_{D}),
matrix size: [(L×N)×(N×12)]=(L×12). Each record in the database can be exclusively reconstructed by the coefficients matrix as follows:

D¹=G_{L}^{T}(Y_{L}+μ_{D}),
matrix size: [(N×L)×(L×12)]=(N×12).
The next steps find common features of the records waveforms, and reduce the records to a small number of coefficients.
9) Use the first F eigenvectors that corresponded to the largest eigenvalues to form the (F×N) matrix G_{F} and a respective (F×12) matrix Y_{F} from the first F rows of Y. The original data D¹ can approximate by:

D̅¹̅=G_{F}^{T}Y_{F}+μ_{D}
matrix size: [(N×F)×(F×12)]=(N×12)
The MSE between the original data D¹ to the approximate data D̅¹̅ is given by the sum of the lowest eigenvalues, starting with F+1:

$$MSE = {\sum }_{i = F + 1}^{L} {λ}_{i}$$

PCA results: Running the PCA on dataset of Human patients using maximum MSE of ˜15% approximates the data with F=1.
10) Use the coefficients matrix from the first segment (Training data D¹) to perform a linear combination from 12 Leads and generate the virtual lead for each segment.

Virtual lead for segment#1=Y_{F}((D¹)^{T}−μ_{D}_{¹})

Virtual lead for segment#2=Y_{F}((D²)^{T}−μ_{D}_{²})

Virtual lead for segment#3=Y_{F}((D³)^{T}−μ_{D}_{³})

Virtual lead dimensions: [(F×12)×(12×N)]=(F×N)

Where in all cases F=1, and we get one virtual Lead for each segment.

The virtual leads (e.g., 3 virtual leads) can then be used in the statistical analysis to estimate the potassium concentration in blood.

In another example method for determining virtual leads, an averaging technique is used. For instance, a mean of 12 leads at each segment, as produced in the PCA process, is another method to generate a virtual lead:

$${μ}_{{D}^{j}} = \frac{1}{12} {\sum }_{i = 1}^{12} {D}_{i}^{j} ( n ) ;$$

$$\{ n = 1 , 2 , … , N \} ;$$

$$\{ j = 1 , 2 , 3 \}$$

Where:

μ_{D}_{^{j}} is the averaged ECG vector of all 12 records (leads) of segment #j.

The 3 virtual leads (from averaging process) are then used in the statistical analysis to estimate the potassium concentration in blood.

Either or both supervised and unsupervised clustering techniques can be used to detect changes in analytes. In some implementations, principal component analysis (PCA) and unsupervised optimal fuzzy clustering (UOFC) can be performed on the three segments of ECG sampled records from human patient under dialysis in order to observe changes in the samples patterns. While in this example PCA and UOFC is employed, other suitable clustering techniques could be employed as well in order to observe changes in the samples patterns. Each segment in the ECG includes 15 records, each record constructed from one minute of ECG filtered and averaged records. The records are represented by N dimensions of samples in the time domain. Each segment includes 15 records which represent a measured potassium concentration. The entire three segments include 45 records in N dimensions, which is the dataset for the clustering analysis. The clustering procedure can include two stages: (1) principal component analysis (PCA) of the records in the set to find the coefficients; and (2) unsupervised optimal fuzzy clustering (UOFC) of the coefficients.

The PCA analysis included the ECG Dataset being expressed in the form of (N×45) ECG matrix as follows:

$${D}^{i} = [ \begin{matrix}{D}_{1} ( 1 ) & … & {D}_{45} ( 1 ) \\ ⋮ & ⋱ & ⋮ \\ {D}_{1} ( N ) & … & {D}_{45} ( N )\end{matrix} ]$$

Where:

N is the number of samples in each record (of 1 minute averaged ECG signal),

A set of basis waveforms (Principal Components) common to all the records are computed as the following process:
1) Calculate the coefficient of D as described in steps 1-9 in the PCA Virtual Lead detection section.
2) These coefficients will be used to divide the records into clusters.

The coefficients matrix Y_{F} is used in the next stage as the features vectors for Unsupervised Optimal Fuzzy Clustering (UOFC) to divide the records into clusters. The UOFC is used in that work can observe changes in the morphology of the ECG during a long period ECG monitoring. The results from the above dataset that UOFC observed changes in the ECG morphology (i.e to observe new cluster) when the potassium measure changed by 0.2 mmol/L. The UOFC performs clustering of data without a priori assumptions about the characteristic features of the clusters. Clustering begins with the assigning of all records to a single cluster and the calculation of memberships in this cluster. Next, the procedure creates a second cluster to include the records with the lowest memberships in the first cluster.

This sequence of adding clusters is repeated until two validity criterions are met.

The validity criterions are based on two parameters:
a) Sum of memberships within each cluster,
b) Standard deviation of members within the cluster.
   Based on these parameters we chose two validity criterions:
a) Partition density
b) Average density.
   The optimal number of clusters in the data set is determined when these criterions are maximal.

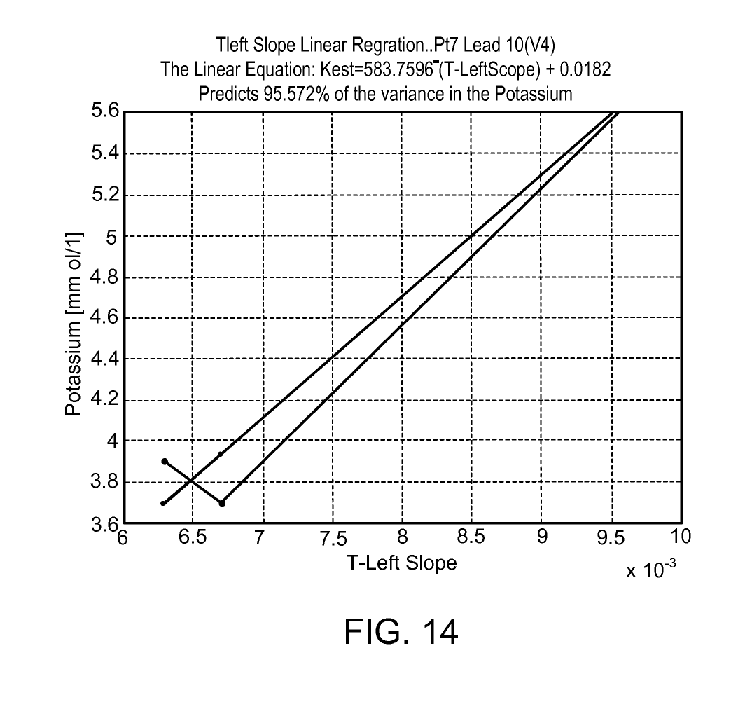

Linear Regression analysis was performed to prove that a relationship between the blood potassium level and the shapes (PQRST complexes) in the ECG signal exists. The Linear Regression process relies on the concept of residuals and on the performance of Data Fitting. Residuals are the difference between the observed values of the response (dependent) variable and the values that a model predicts. When fitting a model, the residuals may be used to evaluate the magnitude of independent random errors. Producing a fit using a linear model requires minimizing the sum of the squares of the residuals. This minimization yields what is called a Least-Squares Fit. In FIG. 14 below, the red dots indicates the measured data and the blue solid line indicate the linear model (Potassium=a*X+b). One measure of the fitting is the Determination Coefficient, or R². It indicates how closely values obtained from fitting a model match the dependent variable the model is intended to predict. The residual variance from the fitted model is:

R²=1−SumSresid/SumStotal

Where:

SumSresid is the sum of the squared residuals from the regression.

SumStotal is the sum of the squared differences from the mean of the dependent variable (total sum of squares).

Both values are positive scalars. Therefore the linear equation Potassium=a*X+b predicts (100*R²) % of the variance in the potassium, where X—is a parameter in the PQRST complex of the ECG.

For parameters that have good results in the linear regression, UOFC can be performed (possibly in combination with PCA) on those parameters to determine whether there have been any relevant changes in potassium values. PCA on ECG waveform analysis can be performed to derive waveform coefficients. Linear regression of those coefficients can also be used to identify changes in potassium levels.

A significant correlation was found between parameters containing the T wave and potassium. High prediction percentage (above 70%) of the variance in the potassium was observed.

In some implementations, the P-wave may be used as a separate or complementary indicator of analyte levels in a patient's bloodstream. The studies have shown that P-wave characteristics, like the T-wave, may also be used to assess potassium levels as the P-wave is also sensitive to changes in potassium levels. For instance, it has been observed that increased potassium levels tend to result in reduced P-wave amplitudes. In some examples, P-wave features can be used confirm assessments of analyte levels determined from T-wave analysis. Thus, if the T-wave change suggests an increase in potassium and the P-wave shows a corresponding change, then there may be higher confidence that the T-wave analysis is accurate. Similarly, if the P-wave and T-wave indicate contrary conclusions, then the confidence of either analysis may be lower.

In some implementations, different forms of analysis may be used based on a type or characteristic of the waveform measured from the patient. For example, using pattern recognition techniques, the shape of the patient's T-wave can be matched to a particular pre-defined shape. Some ECGs may be biphasic, while some may exhibit a single upright T-wave. Some ECGs exhibit bifid showing waves with two or more humps. These various shapes can be recognized, and an appropriate form of analysis selected accordingly. For example, where the T-wave is determined to have a single positive hump, right-sided slope parameters may be used in the analysis. For biphasic, center of gravity techniques may be used, or the signal may be rectified prior to analysis.

It is also noted that in conducting the pig studies, the same pig was used as the subject of each study. Between each study, the pig was observed to gain weight. Accordingly, the data is being considered to determine whether there is a correlation between increases in body mass index (BMI) and the potassium/T-wave relationship. This research may indicate, for example, whether T-waves or other ECG signal components for a patient are more or less sensitive to changes in analyte levels in the patient's bloodstream. The weight or BMI of a patient might then be incorporated into the analysis of the ECG signal for more accurate results.

Other implementations of the techniques described herein for assessing analyte levels from ECG data or other electrical signal data are also contemplated. For example, the ECG data or other electrical signal data may be obtained from implanted sensors or from on-body sensors connected to the patient. Such sensors may include a limited number of electrodes, including down to a single channel (two electrodes) of ECG data. Moreover, electrical information from other use implanted devices such as pacemakers, transvenous defibrillators, subcutaneous defibrillators, or other devices may processed using the techniques described above to estimate potassium (or other analyte) values, or to generate alerts for low or high values without calculating a precise estimate of the parameter.

Moreover, in certain implementations, the system may employ distributed processing techniques. For example, processors associated with one or more of the sensors can process obtained signal data prior to transmitting the processed data to another computing device. For example, a processor that receives signal data from an ECG lead or other sensor can perform PCA to compress the data prior to communicating the data to a mobile computing device or other computing device where the processed data may be analyzed further to assess analyte levels and presented to the user. Compressing the data through PCA prior to sending the data to the mobile or other computing device facilitates data transmission and also can conserve energy at the mobile computing device, for example. Other divisions of processing responsibilities between the sensors and the mobile computing device or other computing device may also be implemented. For example, all processing may occur on a front-end prior to sending data to the mobile computing device or other computing device, or the mobile computing device or other computing device may obtain raw data from the sensors and perform all stages of processing.

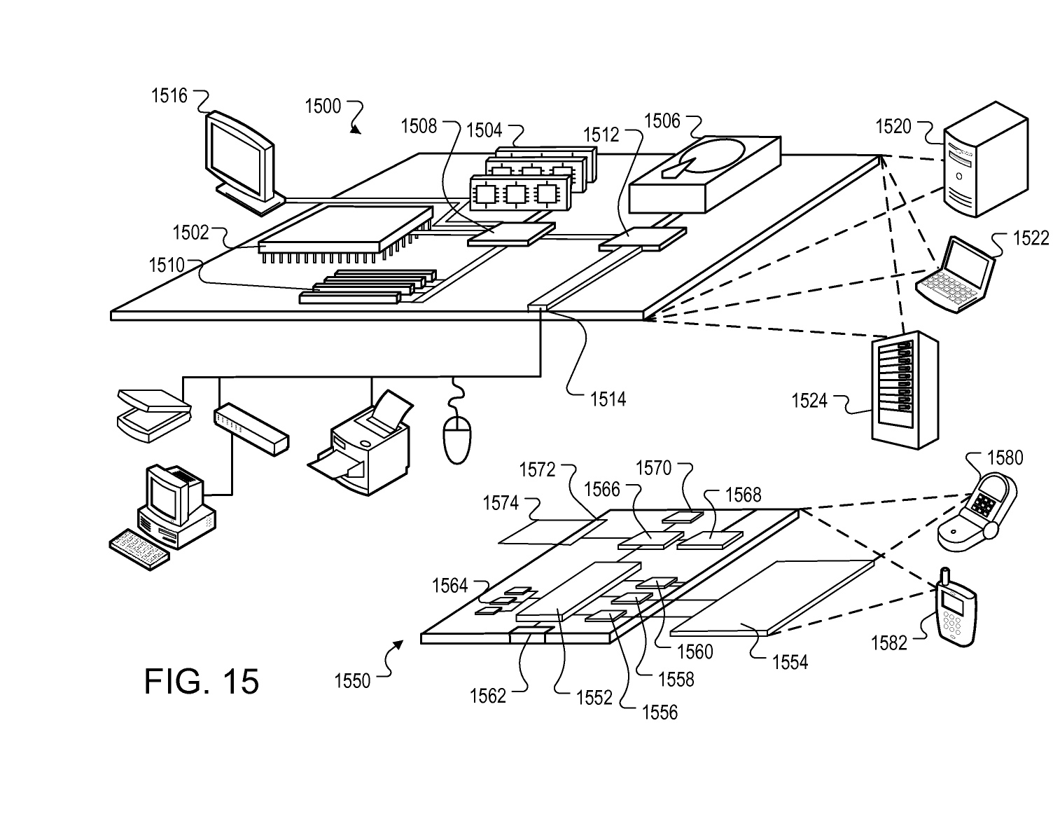

FIG. 15 is a block diagram of computing devices 1500, 1550 that may be used to implement the systems and methods described in this document, as either a client or as a server or plurality of servers. Computing device 1500 is intended to represent various forms of digital computers, such as laptops, desktops, workstations, personal digital assistants, servers, blade servers, mainframes, and other appropriate computers. Computing device 1550 is intended to represent various forms of mobile devices, such as personal digital assistants, cellular telephones, smartphones, and other similar computing devices. Additionally computing device 1500 or 1550 can include Universal Serial Bus (USB) flash drives. The USB flash drives may store operating systems and other applications. The USB flash drives can include input/output components, such as a wireless transmitter or USB connector that may be inserted into a USB port of another computing device. The components shown here, their connections and relationships, and their functions, are meant to be exemplary only, and are not meant to limit implementations described and/or claimed in this document.

Computing device 1500 includes a processor 1502, memory 1504, a storage device 1506, a high-speed interface 1508 connecting to memory 1504 and high-speed expansion ports 1510, and a low speed interface 1512 connecting to low speed bus 1514 and storage device 1506. Each of the components 1502, 1504, 1506, 1508, 1510, and 1512, are interconnected using various busses, and may be mounted on a common motherboard or in other manners as appropriate. The processor 1502 can process instructions for execution within the computing device 1500, including instructions stored in the memory 1504 or on the storage device 1506 to display graphical information for a GUI on an external input/output device, such as display 1516 coupled to high speed interface 1508. In other implementations, multiple processors and/or multiple buses may be used, as appropriate, along with multiple memories and types of memory. Also, multiple computing devices 1500 may be connected, with each device providing portions of the necessary operations (e.g., as a server bank, a group of blade servers, or a multi-processor system).

The memory 1504 stores information within the computing device 1500. In one implementation, the memory 1504 is a volatile memory unit or units. In another implementation, the memory 1504 is a non-volatile memory unit or units. The memory 1504 may also be another form of computer-readable medium, such as a magnetic or optical disk.

The storage device 1506 is capable of providing mass storage for the computing device 1500. In one implementation, the storage device 1506 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 1504, the storage device 1506, or memory on processor 1502.

The high speed controller 1508 manages bandwidth-intensive operations for the computing device 1500, while the low speed controller 1512 manages lower bandwidth-intensive operations. Such allocation of functions is exemplary only. In one implementation, the high-speed controller 1508 is coupled to memory 1504, display 1516 (e.g., through a graphics processor or accelerator), and to high-speed expansion ports 1510, which may accept various expansion cards (not shown). In the implementation, low-speed controller 1512 is coupled to storage device 1506 and low-speed expansion port 1514. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 1500 may be implemented in a number of different forms, as shown in the figure. For example, it may be implemented as a standard server 1520, or multiple times in a group of such servers. It may also be implemented as part of a rack server system 1524. In addition, it may be implemented in a personal computer such as a laptop computer 1522. Alternatively, components from computing device 1500 may be combined with other components in a mobile device (not shown), such as device 1550. Each of such devices may contain one or more of computing device 1500, 1550, and an entire system may be made up of multiple computing devices 1500, 1550 communicating with each other.

Computing device 1550 includes a processor 1552, memory 1564, an input/output device such as a display 1554, a communication interface 1566, and a transceiver 1568, among other components. The device 1550 may also be provided with a storage device, such as a microdrive or other device, to provide additional storage. Each of the components 1550, 1552, 1564, 1554, 1566, and 1568, are interconnected using various buses, and several of the components may be mounted on a common motherboard or in other manners as appropriate.

The processor 1552 can execute instructions within the computing device 1550, including instructions stored in the memory 1564. The processor may be implemented as a chipset of chips that include separate and multiple analog and digital processors. Additionally, the processor may be implemented using any of a number of architectures. For example, the processor 1552 may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 1550, such as control of user interfaces, applications run by device 1550, and wireless communication by device 1550.

Processor 1552 may communicate with a user through control interface 1558 and display interface 1556 coupled to a display 1554. The display 1554 may be, for example, a TFT (Thin-Film-Transistor Liquid Crystal Display) display or an OLED (Organic Light Emitting Diode) display, or other appropriate display technology. The display interface 1556 may comprise appropriate circuitry for driving the display 1554 to present graphical and other information to a user. The control interface 1558 may receive commands from a user and convert them for submission to the processor 1552. In addition, an external interface 1562 may be provide in communication with processor 1552, so as to enable near area communication of device 1550 with other devices. External interface 1562 may provide, for example, for wired communication in some implementations, or for wireless communication in other implementations, and multiple interfaces may also be used.

The memory 1564 stores information within the computing device 1550. The memory 1564 can be implemented as one or more of a computer-readable medium or media, a volatile memory unit or units, or a non-volatile memory unit or units. Expansion memory 1574 may also be provided and connected to device 1550 through expansion interface 1572, which may include, for example, a SIMM (Single In Line Memory Module) card interface. Such expansion memory 1574 may provide extra storage space for device 1550, or may also store applications or other information for device 1550. Specifically, expansion memory 1574 may include instructions to carry out or supplement the processes described above, and may include secure information also. Thus, for example, expansion memory 1574 may be provide as a security module for device 1550, and may be programmed with instructions that permit secure use of device 1550. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 1564, expansion memory 1574, or memory on processor 1552 that may be received, for example, over transceiver 1568 or external interface 1562.

Device 1550 may communicate wirelessly through communication interface 1566, which may include digital signal processing circuitry where necessary. Communication interface 1566 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1568. In addition, short-range communication may occur, such as using a Bluetooth, WiFi, or other such transceiver (not shown). In addition, GPS (Global Positioning System) receiver module 1570 may provide additional navigation- and location-related wireless data to device 1550, which may be used as appropriate by applications running on device 1550.

Device 1550 may also communicate audibly using audio codec 1560, which may receive spoken information from a user and convert it to usable digital information. Audio codec 1560 may likewise generate audible sound for a user, such as through a speaker, e.g., in a handset of device 1550. Such sound may include sound from voice telephone calls, may include recorded sound (e.g., voice messages, music files, etc.) and may also include sound generated by applications operating on device 1550.

The computing device 1550 may be implemented in a number of different forms, as shown in the figure. For example, it may be implemented as a cellular telephone 1580. It may also be implemented as part of a smartphone 1582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

Although a few implementations have been described in detail above, other modifications are possible. Moreover, other mechanisms quantifying potassium based on ECG data may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

What is claimed is:

1. A computer-implemented method, comprising:
accessing, by a computer system, electrogram data for a patient, wherein the electrogram data is obtained using one or more leads that sense physiological electrical activity of the patient;
identifying, by the computer system, one or more waveform features from the electrogram data;
identifying, by the computer system, one or more correlations between values of the one or more waveform features and analyte levels;
determining, by the computer system, one or more estimated analyte levels in the patient based on 1) the one or more waveform features identified from the electrogram data and 2) the one or more correlations; and
outputting, by the computer system, information related to the one or more estimated analyte levels.

2. The computer-implemented method of claim 1, further comprising:
before identifying the one or more waveform features, filtering the electrogram data to generate filtered electrogram data;
wherein the one or more waveform features are identified from the filtered electrogram data.

3. The computer-implemented method of claim 2, wherein the filtering includes a first filtering process comprising:
identifying R peak values in the electrogram data;
identifying intervals in the electrogram data between adjacent R peak values;
determining an average for the intervals;
identifying a portion of the intervals that are at least a threshold value above or below the average; and
removing the portion of the intervals from the electrogram data to generate the filtered electrogram data.

4. The computer-implemented method of claim 3, wherein the vector for the electrogram data comprises a PQRST complex electrogram data vector or any component thereof.

5. The computer-implemented method of claim 3, wherein the threshold value comprises a threshold percentile above or below the average.

6. The computer-implemented method of claim 3, wherein the average for the intervals is determined from only a portion of the electrogram data that is identified within a window of time from the electrogram data.

7. The computer-implemented method of claim 2, wherein the filtering includes a second filtering process comprising:
identifying R peak values for R-waves in the electrogram data;
determining an average R peak value from the identified R peak values;
identifying a portion of the R-waves with R peak values that are at least a threshold value above or below the average R peak value; and
removing the portion of the R-waves from the electrogram data to generate the filtered electrogram data.

8. The computer-implemented method of claim 7, wherein the vector for the electrogram data comprises a PQRST complex electrogram data vector or any component thereof.

9. The computer-implemented method of claim 7, wherein the threshold value comprises a threshold percentile above or below the average R peak value.

10. The computer-implemented method of claim 7, wherein the average R peak value is determined from only a portion of the electrogram data that is identified within a window of time from the electrogram data.

11. The computer-implemented method of claim 2, wherein the filtering includes a third filtering process comprising:
identifying a vector for the electrogram data;
identifying an average ECG vector;
determining a statistical covariance between the average ECG vector and the vector for the electrogram data;
determining one or more correlation coefficients for the electrogram data based on determined statistical covariance; and
removing portions of the electrogram data with corresponding correlation coefficients that are less than a threshold correlation value to generate the filtered electrogram data.

12. The computer-implemented method of claim 11, wherein the vector for the electrogram data comprises a PQRST complex electrogram data vector.

13. The computer-implemented method of claim 2, wherein the filtering includes a fourth filtering process comprising:
for a particular P wave in the electrogram data, identifying at least a threshold number of preceding P waves;
determining a mean voltage level for the preceding P waves;
adjusting the elevation of the particular P wave and portions of the electrogram data surrounding or to the left of the P wave based on the mean voltage level to generate the filtered electrogram data.

14. The computer-implemented method of claim 2, wherein the filtering includes a fifth filtering process comprising:
averaging electrogram data from the one or more leads to generate the filtered electrogram data.

15. The computer-implemented method of claim 1, wherein the one or more waveform features identified from the electrogram data includes a P-wave that precedes an R-wave in the electrogram data.

16. The computer-implemented method of claim 15, wherein the P-wave includes one or more of i) a P-wave area value comprising an area underneath the P-wave and ii) a P-wave amplitude value comprising an amplitude of the P-wave.

17. The computer-implemented method of claim 1, wherein the one or more waveform features identified from the electrogram data includes a QRS complex that comprises Q, R, and S peak points for a Q-wave, an R-wave, and an S-wave.

18. The computer-implemented method of claim 17, wherein the QRS complex includes one or more of i) a QRS area value comprising an area of a triangle formed by the Q, R, and S peak points and ii) a QRS area changes value comprising a change in the QRS area value between one or more R-waves.

19. The computer-implemented method of claim 17, wherein identification of the QRS complex from the electrogram data comprises:
identifying the R peak point for the R-wave in the electrogram data; and
identifying the S peak point for the S-wave and the Q-wave nadir for the Q-wave based on a comparison of a first order derivative of the electrogram data to a statistically defined threshold value.

20. The computer-implemented method of claim 1, wherein the one or more waveform features identified from the electrogram data includes a T-wave that proceeds after an R-wave in the electrogram data.

21. The computer-implemented method of claim 20, wherein the T-wave is divided into sections based on a relationship between i) a peak of the T-wave and ii) a beginning and an end of the T-wave.

22. The computer-implemented method of claim 20, wherein the T-wave includes one or more of i) a T-wave area value comprising an area underneath the T-wave, ii) a T-wave amplitude value comprising an amplitude of the T-wave, iii) a T-wave left slope value comprising a slope value for a left portion of the T-wave, iv) a T-wave right slope value comprising a slope value for a right portion of the T-wave, and v) a T-wave center of gravity value comprising a center point under a curve of the T-wave.

23. The computer-implemented method of claim 22, wherein the T-wave is divided into sections and the following features are determined for each of the sections: the T-wave area value, the T-wave amplitude, the T-wave left slope value, the T-wave right slope value, and the T-wave center of gravity.

24. The computer-implemented method of claim 22, wherein determination of one or more of the T-wave right slope value and the T-wave left slope value comprises:
identifying a start and end point of the T-wave from the electrogram data;
identifying an inflection point at which a second derivative for a curve of the T-wave changes signs;
determine i) a left point that is a threshold number of samples left of the inflection point along the curve of the T-wave and ii) a right point that is a threshold number of samples right of the inflection point along the curve of the T-wave; and
determine a slope between the left point and the right point.

25. The computer-implemented method of claim 22, wherein determination of one or more of the T-wave right slope value and the T-wave left slope value comprises:
identifying a start and end point of the T-wave from the electrogram data;
determine a first derivative between a peak of the T-wave and the end point of the T-wave; and
determine a mean of a plurality of slope value samples that are derived from sample points along the first derivative.

26. The computer-implemented method of claim 22, wherein determination of one or more of the T-wave right slope value and the T-wave left slope value comprises:
identifying a start and end point of the T-wave from the electrogram data;
determine a first derivative between a peak of the T-wave and the end point of the T-wave;
determine a plurality of mean slope values, wherein each mean slope value comprises a mean of a plurality of slope values for sample points along the a curve of the T-wave, the slope values being derived from the first derivative; and
identifying a minimum of the plurality of mean slope values.

27. The computer-implemented method of claim 20, wherein identification of the T-wave from the electrogram data comprises:
selecting a size for a sliding window;
iteratively moving a position of the sliding window forward in time along the electrogram data and, at each iteration, determining an area under a curve defined by the electrogram data; and
identifying starting and ending points for the T-wave based on positions of the sliding window when on a maximum area value and a minimum area value was determined.

28. The computer-implemented method of claim 20, wherein identification of the T-wave from the electrogram data comprises:
determining a line from a T-wave peak point to a heart rate adjusted point forward in time;
evaluating vertical distances between the line and a waveform defined by the electrogram data; and
identifying a point in time on the waveform with a maximum vertical distance as the start or end point of the T-wave.

29. The computer-implemented method of claim 1, wherein the determining of the one or more estimated analyte levels comprises determining a virtual lead that indicates the one or more estimated analyte levels for the patient based on the electrogram data derived from the one or more leads that sense physiological electrical activity of the patient.

30. The computer-implemented method of claim 1, wherein identifying the one or more correlations between values of the one or more waveform features and analyte levels comprises:
transforming a data matrix representing the electrogram data for the one or more leads into a virtual lead space that indicates the one or more estimated analyte levels for the patient, the transformation of the data matrix generating one or more virtual leads that indicate analyte levels for the patient; and
statistically analyzing the one or more virtual leads to identify the one or more correlations.

31. The computer-implemented method of claim 30, wherein the transforming comprises principal component analysis (PCA) for the data matrix.

32. The computer-implemented method of claim 30, wherein the transforming comprises PCA of the data matrix and unsupervised optimal fuzzy clustering of a coefficient matrix generated from the PCA of the data matrix.

33. The computer-implemented method of claim 30, wherein the statistically analyzing comprises performing multiple linear regression or multivariate regression analysis on the one or more virtual leads.

34. The computer-implemented method of claim 1, wherein the analyte levels are selected from the group consisting of: potassium, calcium, magnesium, phosphorous, and anti-arrhythmic drugs.

35. The computer-implemented method of claim 1, wherein the output information identifies one or more ranges that are associated with the one or more estimated analyte levels.

36. The computer-implemented method of claim 1, wherein the output information identifies whether the one or more estimated analyte levels fall within one or more ranges.

37. The computer-implemented method of claim 1, wherein the output information identifies at least a portion of the one or more estimated analyte levels.

38. The computer-implemented method of claim 1, further comprising:
recording, based on electrogram data and corresponding analyte level measurements, the one or more correlations that are specific to the patient.

39. The computer-implemented method of claim 1, further comprising:
generating an mathematically characterized template that is specific to the patient and that provides a baseline of analyte levels for the patient; and
comparing the one or more estimated analyte levels for the patient to the template to identify deviations from the template.

40. The computer-implemented method of claim 1, further comprising:
performing frequency domain analysis with regard to the electrogram data.

41. The computer-implemented method of claim 1, further comprising:
performing a wavelet transform with regard to the electrogram data.

42. The computer-implemented method of claim 1, further comprising:
modeling the electrogram data using a hidden Markov model.

43. The computer-implemented method of claim 1, further comprising:
performing linear discriminate analysis with regard to each characteristic of the electrogram data.

44. The computer-implemented method of claim 1, wherein the electrogram data is obtained from an implanted recording system.

45. The computer-implemented method of claim 44, wherein the implanted recording system comprises a dedicated system for assessing analyte levels.

46. The computer-implemented method of claim 44, wherein the implanted recording system comprises an implantable loop recorder that is capable of being used to diagnose arrhythmia or syncope.

47. The computer-implemented method of claim 44, wherein the implanted recording system is included in a pacemaker, defibrillation, or resynchronization system.

48. The computer-implemented method of claim 44, wherein the implanted recording system comprises an indwelling dialysis catheter.

49. The computer-implemented method of claim 44, wherein the implanted recording system comprises an implant.

50. The computer-implemented method of claim 49, wherein the implant is an abdominal implant, a central nervous system implant, or a vascular implant.

51. The computer-implemented method of claim 44, wherein the implanted recording system comprises an ingestable device.

52. The computer-implemented method of claim 51, wherein the ingestable device comprises an electronic capsule or tablet.

53. The computer-implemented method of claim 1, further comprising determining, based on the electrogram data, a risk that the patient will develop ventricular arrhythmias.

54. The computer-implemented method of claim 1, further comprising determining, based on the electrogram data, a risk that the patient will develop atrial fibrillation.

55. The computer-implemented method of claim 1, further comprising determining, based on the electrogram data, a risk that the patient will experience drug-induced proarrhythmia.

56. The computer-implemented method of claim 1, wherein the computer system comprises a smartphone, a tablet computing device, or a notebook computer.

57. A computer-implemented method comprising:
accessing, by a computer system, electrical signal data for a patient, wherein the electrical signal data is obtained using one or more leads that sense physiological electrical activity of the patient;
identifying, by the computer system, one or more waveform features from the electrical signal data;
identifying, by the computer system, one or more correlations between values of the one or more waveform features and analyte levels;
determining, by the computer system, one or more estimated analyte levels in the patient based on 1) the one or more waveform features identified from the electrical signal data and 2) the one or more correlations; and
outputting, by the computer system, information related to the one or more estimated analyte levels.

58. The computer-implemented method of claim 57, wherein the electrical signal data is selected from a group consisting of electrocardiogram (ECG) data, electroencephalography (EEG) data, and data that characterizes the patient's response to a localized stimulation.

59. The computer-implemented method of claim 1, further comprising determining information that characterizes the patient's body position at a time when the electrogram data is obtained.

60. The computer-implemented method of claim 59, wherein determining the information that characterizes the patient's body position comprises processing signals obtained from an accelerometer connected to the patient.

61. The computer-implemented method of claim 59, wherein the one or more waveform features are identified in response to determining that the patient's body position matches a predetermined body position.

62. The computer-implemented method of claim 59, further comprising determining that the patient's body position at the time when the electrogram data is obtained has changed from a predetermined body position, and in response to determining that the patient's body position has changed from the predetermined body position, adjusting the one or more estimated analyte levels.

63. The computer-implemented method of claim 1, further comprising:
monitoring the patient's heart rate; and
determining that the patient's heart rate is within an acceptable range of a baseline heart rate,
wherein the electrogram data is accessed in response to determining that the patient's heart rate is within the acceptable range.

64. The computer-implemented method of claim 63, wherein the acceptable range is ten beats per minute above or below the baseline heart rate.

65. The computer-implemented method of claim 1, further comprising determining that the patient's heart rate at a time when the electrogram data is obtained deviates from a baseline heart rate, and in response to determining that the patient's heart rate deviates from the baseline heart rate, adjusting the one or more estimated analyte levels.

66. The computer-implemented method of claim 6, wherein the window of time is defined by at least one of a start time and an end time, the start time and end time corresponding to a particular time of day.

67. The computer-implemented method of claim 6, wherein the window of time is determined based on a time when the patient's body position or heart rate matches a baseline body position or a baseline heart rate.

68. The computer-implemented method of claim 29, wherein determining the virtual lead that indicates the one or more estimated analyte levels for the patient comprises determining a difference between adjacent unipolar electrodes in the one or more leads and comparing the difference to a signal from a local bipole.

69. The computer-implemented method of claim 1, further comprising determining a time-based derivative of the electrogram data, wherein the one or more waveform features are identified from the time-based derivative of the electrogram data.

70. The computer-implemented method of claim 39, further comprising generating, based on a determination that the one or more estimated analyte levels for the patient deviate at least a threshold amount from baseline analyte levels in the patient-specific template, an alert to notify a user of the deviation.

71. The computer-implemented method of claim 70, wherein generating the mathematically characterized template comprises drawing blood from the patient and measuring one or more components to determine the baseline of analyte levels.

72. The computer-implemented method of claim 53, wherein determining the risk that the patient will develop ventricular arrhythmias comprises determining a center of gravity or a T-wave slope based on the patient's electrogram data.

73. The computer-implemented method of claim 1, wherein the electrogram data comprises one or more of electrocardiogram data, brain electrogram data, muscular electrogram data, myoelectrogram data, and neuro-electrogram data.

74. The computer-implemented method of claim 1, wherein the one or more leads that sense physiological electrical activity of the patient are physically attached to the patient.
